(19) United States
(12) Patent Application Publication
(10) Pub. No.: US 2023/0390577 A1
(43) Pub. Date: Dec. 7, 2023

(54) SYSTEM FOR PERFORMING A TRANSCUTANEOUS PHOTODYNAMIC THERAPY (PDT) IN AN ORGAN OR ORGAN SEGMENT OF AN ORGANIC BODY

(71) Applicant: Richard Wolf GmbH, Knittlingen (DE)

(72) Inventors: Bernd Claus WEBER, Karlsruhe (DE); Stephan PRESTEL, Rheinstetten (DE); Giacomo RIECKERT, Knittlingen (DE)

(21) Appl. No.: 18/327,954

(22) Filed: Jun. 2, 2023

(30) Foreign Application Priority Data
Jun. 3, 2022 (DE) 10 2022 205 706.6

(51) Int. Cl. A61N 5/06

(52) U.S. Cl. CPC A61N 5/062; A61N 2005/0612; A61N 2005/0651

(57) ABSTRACT

A system (1) performs a transcutaneous photodynamic therapy (PDT) in an organ (3)/organ segment of an organic body (5) and includes light applicators (9), alight applicators light/power supply unit, a light applicators placement template (39) for a defined orientation, positioning and fixing, and a support point element (100). The light applicators have an insertion section (15) for transcutaneous piercing along a piercing axis (E) into the organ/organ segment, a light-emitting applicator tip (19) at the distal end (13) of the insertion section and a defined fixing point (43) at a proximal distance d from the light-emitting applicator tip. The distance d is identical for each of the individual light applicators. The support point element defines a positionable support point (93), coaxial with the piercing axis, for laterally supporting the respective light applicator. A distal distance f of the support point from the placement template is adjustable.

### CROSS REFERENCE TO RELATED APPLICATIONS

This application claims the benefit of priority under 35 U.S.C. § 119 of German Application 10 2022 205 706.6, filed Jun. 3, 2022, the entire contents of which are incorporated herein by reference.

### TECHNICAL FIELD

The present disclosure relates to a system for performing a transcutaneous photodynamic therapy (PDT) on a body or organ segment of an organic body.

### BACKGROUND

PDT is a known medical treatment of pathological tissue of a patient. For this, the patient is administered a photosensitiser or marker agent which selectively accumulates on the pathological tissue to be treated. In PDT, light is then applied by means of a light applicator or several light applicators directly onto or even into the pathological tissue, in order to encourage the light-induced formation of oxygen radicles by way of the locally restrictedly concentrated photosensitiser or marker agent and thereby destroy the pathological tissue, such as a tumour. To do this, laser light is typically coupled in and directed to the tissue. If the pathological tissue is arranged flat on an outer surface of the body, e.g. the skin, or an inner surface, e.g. the inner surface of the oesophagus or the walls of the intestine, the therapy light can be relatively easily decoupled and shone onto the pathological tissue surface. However, if the pathological tissue extends over a volume in an internal organ or organ segment, due to the limited penetration depth of the light into the tissue, a tumour cannot always been effectively irradiated from “outside”. In this case, PDT is particularly effective if the light is irradiated from inside the pathological tissue volume. For this, the light applicator or the light applicators must be pierced into the pathological tissue. This is also known as interstitial (through the inner surfaces) and/or transcutaneous (through the skin) PDT. U.S. Pat. No. 6,048,359 describes, for example, a system for performing transcutaneous PDT, in order to irradiate an internal volume in the body of a patient. Here, a plurality of needle-like (needle shaped) light applicators are pierced in parallel through the skin into the body, wherein each light applicator comprises lateral light decouplers distributed over its length in order to irradiate a volume in the body.

A disadvantage of the known solution is that, firstly the light applicators have to be relatively thick in configuration and secondly, they are relatively complex and expensive, so that on the one hand, particularly in total, they are relatively burdensome for the patient, and, on the other hand they cannot be produced as a disposable article for single use. With thinner light applicators there is a risk that they could buckle when piercing the skin and therefore the applicator tip cannot be positioned accurately enough in the body.

This results in the task of providing a more cost-effective system for performing a transcutaneous PDT with thinner light applicators that are more gentle on the patient, wherein the system should allow exact positioning of the applicator tips in the body and thereby seamless irradiation of the organ or organ segment.

In this respect, the present disclosure relates to a further development of DE 10 2021 211 328 A1 and DE 10 2021 211 331 A1 that are still to be published in the future.

### SUMMARY

According to a first aspect of the present disclosure, in order to solve this problem a system for performing a transcutaneous PDT in an organ or organ segment of an organic body is provided, in which the system comprises a plurality of individual manually positionable light applicators, a supply unit for supplying the light applicators with light and/or power, a placement template placeable in a defined position in relation to the organic body, for the defined alignment, positioning and fixing of the individual light applicators in or on the placement template and thereby ultimately for the defined placement of the light-emitting applicator tip in the organ or organ segment, and a support point element. For this, the individually placeable light applicators each have a needle-like (needle shaped) insertion section for transcutaneous piercing along a piercing axis into the organ or organ segment, a light-emitting applicator tip at the distal end of the insertion section and at least one defined fixing point at a proximal distance d from the light-emitting applicator tip. Here, the distance d is identical for each of the individual light applicators, wherein the support point element for each of the individual light applicators defines with the piercing axis a coaxially positioned or positionable support point for the lateral supporting of the respective light applicator, wherein a distal distance f of the support point from the placement template is adjustable.

Through the support point element arranged at the lateral distance f from the placement template, the risk of buckling of the individual light applicators on piercing can be essentially reduced. It can also be said that through the support point element, the individual light applicators have to be less rigid, i.e. they can be configured more thinly and cost-effectively. Piercing is therefore less invasive. In addition, through the adjustability of the distance f, it is possible, in different cases of application, to use only one form of light applicator, i.e. the distance d is identical for all light applicators, regardless of the application. Therefore, different lengths of light applicators for different cases of application do not have be at hand and correctly selected in accordance with the case of application. After the placement template has been placed and fixed in a defined position in relation to the body, a target point for placement of the application tip in the body is defined. The support point element can then be moved as far as possible to the body, for example until it comes into contact with the skin, in order achieve as large a distance f as possible. Through the support point placed therewith, the risk of buckling of the light applicators is greatly reduced. It is also conceivable to first place the support point element straight into its final position—preferably as close as possible to the body or on the skin—and fix it in this position, and only then bring the placement template into the corresponding distance f from the support point element and thus the defined, final position in relation to the organic body.

Optionally, the support point can be formed by an opening in the support point element, through which the light applicator can be passed in a precisely fitting manner.

Optionally, at least along the piercing axis the support point element can be moveably mounted and lockable in position relative to the placement template, or in the case of final placement and fixing of the support point element close to the body prior to final placement of the placement template—inversely the placement template can moveably mounted and lockable in position along the piercing axis relative to the support point element.

Optionally the support point element can be coupled to the placement template guided in such a way that relative to the placement template, the support point element is only linearly moveable along the piercing axis, or in the case of final placement and fixing of the support point element close to the body prior to final placement of the placement template—inversely the placement template can be coupled to the support point element guided in such a way that the placement template is only linearly moveable along the piercing axis.

Optionally the support point element can be configured as a perforated plate with holes, wherein a hole defines a support point, wherein the holes are arranged or arrangeable coaxially to light applicator holders of the placement template.

Optionally the support point element can comprise a skin application surface facing away from the placement template, wherein the skin application surface can be placed on the skin of an organic body and fixed in position there. The skin application surface can, for example, at least partially be an adhesive surface with which the support point element can be stuck onto or adheres to the skin for the duration of the treatment. Preferably, the skin application surface is only sticky or adherent in separate areas arranged around the support points in order to minimize the adhesive area and facilitate subsequent removal from the skin.

Optionally, the support point element can also be moveably positionable relative to the placement template in a plane perpendicular to the piercing axis or—in the case of final placement and fixing of the support point element close to the body prior to final placement of the placement template—inversely the placement template can also be moveably positionable relative to the support point element in a plane perpendicular to the piercing axis.

Optionally, the light applicators can independently of each other be placed in the placement template and the support point element.

Optionally, the placement template can comprise at least one first template part, which defines a first sub-group of fixing point holders, at least one second template part, which defines a second sub-group of fixing point holders, wherein the first template part is displaceable relative to the second template part in a guided manner along the piercing axis.

Optionally, the first sub-group of fixing point holders can define a first fixing point holder partial grid and the second sub-group of fixing point holders a second fixing point holder partial grid, wherein the second fixing point holder partial grid is offset perpendicularly to the piercing axis by a grid offset relative to the first fixing point holder partial grid.

Optionally, the first sub-group of fixing point holders, the second sub-group pf fixing point holders as well as the light applicators can together form a three-dimensional fixing point grid structure, wherein the fixing point grid structure corresponds to a virtual organ-specific target point grid structure for the light-emitting applicator tips in the organ or organ segment and is parallel displaced to the target point grid structure by a distance d along the piercing axis, wherein each light applicator has at least one defined fixing point at a distance d from the light-emitting applicator tip.

Optionally, the second template part can be displaceable in the proximal direction relative to the first template part only up to the stop on the first template part, wherein when the second template part is in contact with the first template part, the first sub-group of fixing point holders defined by the first template part is located distally of the second sub-group of fixing point holders defined by the second template part.

Optionally, the first template part can comprise a first sub-group of fixing point holders of a first fixing point holder grid area, and the second template part a second sub-group of fixing point holders of a second fixing point grid area.

Optionally the light applicators can each have one fixing point which is formed by a stop and is lockable.

Preferably, with the above-described assembly, with the realization and positioning of the placement template outside the body in a first step, it is endeavored to show a virtual three-dimensional target point grid structure by means of a parallel displacement or translation about distance d on a three-dimensional fixing point grid structure. On the one hand, the virtual three-dimensional target point grid structure in the body of the patient represents the defined arrangement of the light-emitting applicator tips in the organ or organ segment with a view to seamless irradiation of the organ or organ segment. The operator should therefore fill the target point grid structure with the light-emitting applicator tips. However, the target points of the target point grid structure in the patient's body are not visible to the operator and cannot therefore be accessed specifically and directly with the light-emitting applicator tips. The fixing point grid structure, formed by the placement template, which is represented by the fixing point holders in or on the placement template outside the patient's body, is, in contrast to the virtual target points of the target point grid structure lying within the body, visible to the operator and is specifically and directly accessible with the at least one fixing point of a respective light applicator. After the first step of parallel displacement or translation, in a second step the light-emitting applicator tips are therefore placed in a more defined, clearer and thereby safer way on the virtual target points of the target point grid structure in the organ or organ segment that are invisible to the operator, by means of a parallel displacement that is inverse in relation to the above-described parallel displacement. Particularly in the case of greater distances between the placement template and the skin of the patient, the support point element placeable distally of the placement template at adjustable distance f then reduces the risk of the thin light applicators buckling during piercing into the skin.

In a first step, the virtual, not visible and therefore not directly accessible organ-specific target point grid structure within the body, can be transformed or shown outside the body by way of a parallel displacement or translation into or onto the fixing point grid structure, represented by the actually existing visible and therefore directly accessible fixing point holders of the placement template.

After this first step, there follows as a second step the positioning of the support point element at the greatest possible distance f distally of the placement template or at the smallest possible distance from the skin of the patient. The inverse procedure is also conceivable, i.e. prior to the placement of the placement template, the placement and fixing of the support point element at the smallest possible distance from the skin and the subsequent placement and fixing of the placement template at a corresponding distance f from the support point element. The operator then carries out the actual therapy preparation, which consists in the distribution and placement of the individual light applicators or the light-emitting applicator tips in the patient with a view to seamless irradiation of the organ or organ segment. In this second step, —starting from the visible and directly accessible fixing point images holders of the placement template outside the body—the individual light-emitting applicator tips are transformed, or moved, by way of parallel displacement which is inverse to the parallel displacement of the first step, in a defined, clear and therefore safe manner to the invisible target points inside the body. Definiteness and unambiguity in the placement of the individual applicator tips in the target points is achieved in that, firstly, the placement template is provided with guides in parallel orientation to each other for defined, unambiguous parallel orientation of the individual light applicators, and, secondly, in that on the one hand the placement template is provided with fixing point holders, and on the other hand, the light applicators are provided with at least one fixing point, with which the individual light applicators can be fixed in or on the fixing point holders of the placement template at a defined and unambiguous point, through which a defined and unambiguous penetration depth of the individual light applicators in the organ or organ segment is achieved. Here, the light applicators are held in the respective support points of the support point element in addition to the guides of the placement template. Through the set distance f, the risk of buckling of the individual light applicators is greatly reduced.

The light applicators can thus be configured particularly thinly and rigidly as a metal shaft and/or tube as only the applicator tip has to be light-emitting. At the applicator tip a light-emitting component in the form of a light guide decoupler or an LED can be arranged for example. The applicator tip can be arranged as a light-transparent diffuser distally of the light-emitting component. The system for performing a transcutaneous PDR is thus considerably more gentle on the tissue and less invasive than systems known from the prior art. Through the three-dimensional fixing point grid structure arising through the aforementioned parallel displacement by length d along the piercing axis from the target point grid structure, with on the other hand fixing point holders arranged in accordance with this three-dimensional fixing point grid structure, and, on the other hand guides, which both together (fixing point holder and guide) and in combination with the at least one light applicator fixing point, allows a movement and final placement of the individual light applicators and the light applicator tips in the body and in the organ exclusively in accordance with a parallel displacement inverse to the aforementioned parallel displacement, and in this sense reduces the related degrees of freedom of the operator to a minimum, it is ensured—in combination with the placement close to the body of the support point element, which in accordance with the invention is adjustable and in this way guide-reinforcing—that the individual light-emitting applicator tips are arranged in accordance with the target point grid structure in the organ or body so that the organ or organ segment is seamlessly irradiated over the entire volume of the target point grid structure. Each grid point of the target point grid structure is preferably occupied by an applicator tip. However, this does not necessarily have to take place at the same time. Particularly rapid performance of the transcutaneous PDT is achieved in that all grid points of the target point grid structure are each occupied by one applicator tip and then the entire organ or organ segment is irradiated at the same time. Alternatively, the transcutaneous PDT can be performed successively for different grid points or grid point group of the target point grid structure. In this way, while accepting a longer treatment period, the number of required light applicators and/or piercings can be reduced, through which the burden on the patient and the costs of the treatment may be able to be reduced.

For example, the target point grid structure can have several target point grid areas that have a grid area distance k along the piercing axis. The target point area can extend flat as target point grid planes or curved as part of the target point grid structure. The percutaneous PDT can then be performed successively for one target point grid surface after the other. For example, an operator can begin with a target point grid area located at the deepest point and then, through corresponding proximal pulling out of the individual light applicators by one grid area distance k each time, work on one target point grid area after the other until the entire target point grid structure has been irradiated and thus the organ or organ segment has been seamlessly irradiated.

The virtual organ-specific target point grid structure lies inside the organic body distributed over the organ or organ segment and is therefore not visible to the operator. The three-dimensional fixing point grid structure, on the other hand, which is preferably defined by a combination of the placement template with its fixing point holders and the individual light applicators with their fixing points, is outside the body and visible to the operator and ensures—in combination with placement close to the body of the invented adjustable, and in this way guide-reinforcing, support point element, —that the individual light-emitting grid applicator tips are then located in the corresponding grid points of the target point grid structure when the fixing point of the respective light applicator is located at the appurtenant grid point of the fixing point grid structure, i.e. in the corresponding fixing point holder or the placement template.

An operator can, for example, by way of a guide, which is firstly centrally located in the placement template and, secondly, its appurtenant fixing point holder is arranged largely distally in the placement template, pierce a first light applicator into the organ or organ segment. Preferably, the placement template, the support point element distally upstream of the placement template and the skin of the patient are initially at a minimal distance with regard to each other. In addition, the support point element is placed with regard to the placement template in such a way that its support points are arranged coaxially to the corresponding light applicator holders on the placement template. By means of an imaging procedure, for example by means of ultrasound sonography, the operator can place the applicator tip of this first light applicator at a grid point of the target point grid structure corresponding to this fixing point holder in the organ or organ segment, i.e. also largely distally and within this selected, distal target point grid structure plane also in a centrally arranged manner. The initial minimal distance of the placement template, support point element and skin relative to each other, and the thus achieved best possible guiding of the light applicator when piercing the skin, ensure that when being introduced into the body, or through the skin of the patient, the first light applicator cannot become buckled. The body of the patient is thereby preferably fixed relatively to a solid reference surface, for example a treatment table. With the correctly positioned first light applicator or light-emitting applicator tip, and maintenance of this selected light applicator position, the placement template can be moved along the piercing axis relative to this and positioned in such a way that the fixing point of the first light applicator is located at the corresponding grid point of the fixing point grid structure. In the case of the above-described procedure with initially minimal distance between the three components placement template, support point element and skin, this takes place in that the placement template is retracted in the proximal direction away from the skin. In this position, both the fixing point of the light applicator on the fixing point holder of the placement template as well as the placement template, can be fixed on the other reference surface, for example a treatment table.

With this procedure, the three-dimensional fixing point grid structure is spatially determined in relation to the body of the patient. Furthermore, with this action, through which the defined distance d between the virtual target point grid structure and fixing point grid structure is brought about, the first step in this procedure, namely the first representation, i.e. the parallel displacement or translation of the virtual target point grid structure in the organ or organ segment within the human body in an area outside the human body is completed.

It now preferably ensured that the support point element—in contrast to the newly positioned placement template at the corresponding distance from the skin of the patient—also remains at a minimal distance from the skin of the patient when the remaining light applicators are individually introduced into the body in order to also ensure optimum guiding for these remaining applicators (no buckling) when piercing the skin. Either the minimum distance of the support point element from the skin is already maintained during retraction of the placement template in the proximal direction. In the other case that, due to the configuration the support point element is initially retracted in the proximal direction together with the placement template, the support point element is then moved back in the distal direction to its original position close to the skin of the patient.

The individual further light applicators can now be placed simply, without error and quickly as they only have to be pushed in through the corresponding guide of the placement template and the respective support point of the support point element without buckling and advanced so far until their fixing point is in the corresponding grid point of the fixing point grid structure, i.e. in the fixing point holder of the placement template. The applicator tip is then automatically situated at the appurtenant grid point of the target point grid structure. Placement of the support point element at a maximal distance from the placement template or minimal distance from the skin of the patient, ensures that when being introduced through the skin, the individual applicators do not become buckled and thereby the appurtenant grid points of the target point grid structure are precisely hit, even if the placement template is at a greater distance from the skin. The virtual organ-specific target point grid structure ensures that on the one hand the individual applicator tips are not too far from each other so that no irradiation gaps occur in the organ or organ segment, and on the other hand are not too close to each other to encumber the patient with unnecessarily many piercings. The target point grid structure is organ-specific as the external shape and size of the target point grid structure depends on the organ or organ segment. In addition, the density of the target point of the target point grid structure depends on how great the penetration depth of the light into the tissue of the respective organ or organ segment is. The smaller the penetration depth of the irradiation light, the denser the target grid structure must be selected, in order to ensure seamless irradiation of the organ or organ segment.

Optionally the supply unit can be set up to supply the light applicators with power, wherein every light applicator has, at the distal end of the insertion section, a power-operated LED and/or another light-emitting component. Compared to a light guide decoupler, an LED and/or another corresponding light-emitting component on the distal end of insertion section has the great advantage that no expensive laser is required, the light of which is coupled via the supply into the light guide. As the supply unit only has to supply the individual light applicators with power, it can be particularly simply and cost-effectively configured. The LED and/or other light-emitting component preferably has a luminous spectrum matched to the photosensitizer or marker substance. Alternatively, or additionally, a light filter can be used for this.

Optionally the fixing point holders are each arranged in or on the appurtenant guide or are formed thereby.

Optionally the individual light applicators can each have at least one fixing point, that is formed by a stop and in accordance with the three-dimensional fixing point grid structure is lockable on a fixing point holder of the placement template. Through the stop and locking, an operator receives haptic, acoustic and/or optical feedback about the correct placement of the fixing point on a grid point of the fixing point grid structure or the corresponding fixing point holder.

Optionally, the virtual organ-specific target point grid structure can comprise a plurality of target points arranged spatially distributed on target point grid areas, wherein the target point grid areas are at a distance k from each other along the piercing axis. Preferably the target point grid areas extend perpendicularly to the piercing axis.

Optionally the individual light applicators can each have at least two fixing points, which are at a distance k from each other along the piercing axis, which is also the distance the target point grid areas are from each other along the piercing axis. Through this, the operator has the option of selecting one of the fixing points and fixing at the corresponding grid point of the fixing point grid structure or the appurtenant fixing point holder of the placement template and thereby determine the fixing point grid area in which the applicator tip should lie.

Optionally, the virtual organ-specific target point grid structure can comprise a plurality of target points arranged spatially distributed on target point grid areas, wherein the target point grid areas are at a distance k from each other along the piercing axis. By selecting the fixing point holder in which the fixing point it to be locked, the operator has the possibility of determining the target point grid area in which the applicator tip is to lie.

Optionally, the virtual organ-specific target point grid structure can comprise a plurality of target points arranged spatially distributed on target point grid areas, wherein at least eight of the target points form corner points of a grid elementary cell of the target point grid structure in the form of a parallelepiped. Preferably, the grid elementary cell has three grid elementary cell edges and four grid elementary cell diagonals, of which one or none extends along the piercing axis. The variant in which none of the three grid elementary cell edges or four grid elementary cell diagonals run along the piercing axis, has the advantage that several target point areas can simultaneously be occupied with applicator tips and thereby the PDT can be performed more quickly. In contrast, the variant in which one of the three grid elementary cell edges or four grid elementary cell diagonals run along the piercing axis, has the advantage that one piercing can be used for several target grid areas and therefore on the one hand fewer piercings are needed, which is gentler on the patient' tissues and means that fewer light applicators are needed, as a result of which the system is made more cost-effective.

According to a second aspect of the present disclosure, that is preferably combinable with the first aspect or is independent of it, the system for performing a transcutaneous PDT in an organ or organ segment of an organic body is provided, wherein the system comprises a plurality of individual light applicators, a supply unit for supplying the light applicators with light and/or power and a placement template placeable at a defined position in relation to the organic body for the defined orientation, placement and fixing of the individual light applicators. The individually placeable light applicators each have a needle-like insertion section for transcutaneous piercing along a piercing axis into the organ or organ segment and a light-emitting applicator tip at the distal end of the insertion section and at least one defined fixing point at a proximal distance d from the light-emitting applicator tip. Furthermore, the placement template defines a plurality of fixing point holder, by means of which the individual light applicators are fixable with their at least one defined fixing point. The placement template can define a plurality of guides, wherein through each of the guides an insertion section of one of the light applicators can be transcutaneously pierced into the organ of the organ segment along the piercing axis. The fixing point holders can be formed on or in the guides.

According to the second aspect, the placement template comprises at least one first template part, which defines a first sub-group of fixing point holders, at least one second template part, which defines a second sub-group of fixing point holders, wherein the first template part is displaceable relative to the second template part in a guided manner along the piercing axis.

By way of the template parts, various sub-groups of light applicators concerted together by means of fixing in corresponding sub-groups of fixing point holders can be optionally moved or held fast. In this way it is possible to hold the organ or organ segment fast or maintain its shape with one sub-group of light applicators, while the other sub-group is being moved.

Such a system and such a procedure can be advantageous for the following reason. If a light applicator inserted into the organ or organ segment is moved or displaced, this then exerts a frictional force on the organ or organ segment and/or organ tissue that is in contact with the light applicator. If the light applicators are all jointly or simultaneously moved in one direction, then the sum of these frictional forces acting on the organ or organ segment in this direction is also correspondingly large. Depending on the various constraints set out below, two in principle different, relevant cases can come about that can also occur in combined form.

In a first case, which is based on the constraint that during the joint and simultaneous movement of the light applicators, the jointly acting total frictional forces in a direction towards the organ or organ segment is greater than the binding force acting within the surrounding tissue directly connected to the organ or organ segment to be treated and in turn smaller than the binding forces acting within the organ or organ segment itself, the organ or organ segment can be “carried along” unchanged as a whole by the light applicators being jointly or simultaneously moved in a joint direction, i.e. during the joint or simultaneous displacement of the light applicators in one direction under the aforementioned constraints, the organ or organ segment keeps its shape but changes its position in the body. In this first case, although the target point grid structure would remain unchanged in shape and therefore still correspond to the shape of the fixing point grid structure, the position of the target point grid structure would change in relation to the still unchanged position of the fixing point grid structure, i.e. the fixing point grid structure would undesirably no longer be displaced in a parallel manner by the original length d which characterizes parallel displacement and corresponds to the distance d between the applicator tip and fixing point.

In a second case, which is based on the constraint that during the joint and simultaneous movement of the light applicators, the jointly acting total frictional forces in a direction towards the organ or organ segment is greater than the binding force acting within the surrounding tissue directly connected to the organ or organ segment to be treated and in turn smaller than the binding forces acting within the organ or organ segment itself, the organ or organ segment can be expanded or stretched in the direction of movement of the light applicators, i.e. during the joint or simultaneous displacement of the light applicators in one direction under the aforementioned constraints, in a first approximation the organ or organ segment keeps its position in the body, but changes its shape. In this second case, the position of the target point grid structure in relation to the position of the fixing point grid structure would in a first approximation be unchanged, i.e. the distance between the two grid structures would, after the joint or simultaneous displacement of the light applicators, in a first approximation still correspond to length d characterizing the parallel displacement, but the shape of the target point grid structure would change in the same way as the shape of the organ and no longer correspond to the shape of the fixing point grid structure.

In both cases, or a combination thereof, such a “stacking” effect would be negative, as seen in mathematical-geometric terms, in both cases the original parallel displacement by length d, through which the fixing point grid structure emerged from the target point grid structure and which forms the basis of the procedure shown here for complete and above all precise treatment of the organ or organ segment, would be impaired or modified, e.g. characterizing parameters of the parallel displacement (object shape, displacement length d) would be changed and therefore even, and above all, seamless irradiation would no longer be automatically guaranteed.

With an increasing number of light applicators, both mentioned cases of the “stacking effect” or its effects become more important as each additional light applicator, at its contact surface with the organ or organ segment, which is essentially formed by the light applicator shaft, exerts an additional static frictional force on the organ or organ segment, which further increases the overall static frictional force. In this way, with an increasing number of light applicators, the probability increases that the overall static fictional force will either exceed the binding forces within the tissue surrounding and directly connected to the organ (first case described above) and/or exceed the binding forces within the organ or organ segment itself (second case described above), thereby resulting either in a displacement of the organ or organ segment in the proximal direction (first case described above) and/or a deformation of the organ or organ segment (second case described above).

The “stacking effect” can be prevented in that one sub-group of light applicators is kept in place while the other is moved or displaced or pulled out. The respective sub-groups can be alternately moved, while the other is held fast. The joint movement and joint holding of the light applicators can be brought about by corresponding movement or holding of the template parts.

Optionally, the first sub-group of fixing point holders can define a first fixing point holder partial grid and the second sub-group of fixing point holders a second fixing point holder partial grid, wherein the second fixing point holder partial grid is offset perpendicularly to the piercing axis by a grid offset relative to the first fixing point holder partial grid. In this way, each light applicator preferably has another light applicator as a laterally adjacent neighbor which is in the respective other sub-group of guides. In this way the “stacking effect” can be particularly well prevented as the resulting static frictional force of neighboring light applicators cancel each other out in pairs and the overall static frictional force is therefore zero. In addition, local displacements and deformations of the organ or organ segment can be prevented. Preferably the first fixing point holder partial grid has approximately the same number of fixing point holders as the second fixing point holder partial grid.

Optionally, the lateral grid offset can be smaller than a side length of a grid elementary cell of the first fixing points holder partial grid and/or the second fixing point holder partial grid. Preferably the side length of a grid elementary cell of the first fixing point holder partial grid and the second fixing point holder partial grid is identical and the lateral grid offset is half the side length of a grid elementary cell. The grid offset can preferably be provided in both orthogonal lateral directions, so that a fixing point holder of the first fixing point holder partial grid forms the midpoint of a grid cell of the second fixing point holder partial grid and vice-versa.

Optionally, the first fixing point holder partial grid and the second fixing point holder partial grid can lie in one fixing point holder grid area. In this way the fixing point holder partial grids supplement each other to form an entire fixing point holder grid in one fixing point holder grid area. Such an arrangement—in comparison with the arrangement described below—has the advantage that when moving or displacing a template part, which represents one of the two fixing point holder partial grids, the frictional forces acting in opposite directions are even better cancelled out as the shaft lengths determining the frictional forces within the organ or organ segment are identical or similar as for at least for a time they protrude in pairs equally far into the organ or organ segment. The striven for pair-wise cancelling out of the frictional force is initially in a simplified manner based on the fact that both fixing point holder grid areas are occupied by the same number of applicators. However, if the fixing point holder partial grids were not in the same fixing point holder partial grid area, it could be that the frictional forces no longer completely compensate each other in a pair-wise manner as the respective shaft lengths located within the organ or organ segment differ. This can be fully or partially compensated for in that the fixing point holder partial grid or the corresponding template part that is initially moved in the proximal direction is occupied by a greater number of light applicators.

Optionally, the first sub-group of guides and fixing point holders, the second sub-group of guides and fixing point holders as well as the light applicators can together form a three-dimensional fixing point grid structure, wherein the fixing point grid structure corresponds to a virtual organ-specific target point grid structure for the light-emitting applicator tips in the organ or organ segment and is parallel displaced to the target point grid structure by a distance d along the piercing axis, wherein each light applicator has at least one defined fixing point at a distance d from the light-emitting applicator tip.

Optionally the supply unit can be set up to supply the light applicators with power, wherein every light applicator can, at the distal end of the insertion section, have a power-operated LED and/or another light-emitting component. Compared to a light guide decoupler, an LED on the distal end of insertion section has the great advantage that no expensive laser is required, the light of which is coupled in via the supply unit into the light guide. As the supply unit only supplies the light applicator with power, it can be particularly simple and cost effective to design. The LED preferably has a luminous spectrum matched to the photosensitizer or marker substance. Alternatively, or additionally, a light filter can be used for this.

Optionally, the individual light applicators can each have at least one fixing point which is formed by a stop and is lockable. Through the stop and locking, an operator receives haptic, acoustic and/or optical feedback about the correct placement of the fixing point on a grid point of the fixing point grid structure or the corresponding fixing point holder of the placement template or template part.

Optionally, the virtual organ-specific target point grid structure can comprise a plurality of target points arranged spatially distributed on target point grid areas, wherein the target point grid areas are at a distance k from each other along the piercing axis. Preferably the target point grid areas extend orthogonally to the piercing axis.

Optionally, at least eight of the target points can form corner points of a grid elementary cell or the target point grid structure in the form of a parallelepiped. Optionally, the grid elementary cell, can have three grid elementary cell edges and four grid elementary cell diagonals, of which one runs along the piercing axis. Through this it is particularly simple to simultaneously position all light applicators guided in the first template part through displacement of the first template part in such a way that the respective light-emitting applicator tips are guided from one target point grid area to the next. During this, the light applicators guided in the second template part are kept in place and thereby keep the organ or organ segment in position and/or suppress its deformation while the light applicators guided in the first template are jointly moved. As soon as the light applicators guided in the first template part are placed, they can be kept in position, whereas the light applicators guided in the second template part are moved with their light-emitting applicator tips to the next target point grid area. Preferably this process starts with target point grid area of the respective template part lying deepest in the body and work is performed by pulling back the respective template part proximally to the next target point grid area. The PDT can in each case be performed when the applicator tips are positioned in their target point in the target point grid.

Optionally, a first sub-group of target points corresponding to a first sub-group of guides or fixing point holders can define a first target point partial gride, and a second sub-group of target points corresponding to a second group of guides or fixing point holders can define a second target point partial grid so that the first target point partial grid and the second target point partial grid supplement each other to form one of the target point areas. In this way, each target point grid area can be completely filled with light-emitting applicator tips in that initially one target point partial grid is filled with light-emitting applicator tips and then the other is filled. This can take place in a simple way through corresponding displacement of the template relative to each other.

Optionally, the individual light applicators can each have at least two fixing points which along the piercing axis are at a distance k from each other, which is also that of the target point grid areas from one another. Through this, the operator can, through one of the fixing points which is to be locked in the grid point of the fixing point grid structure or in the corresponding fixing point holder of the respective template part, determine in which target point grid area the light-emitting applicator tips should lie.

Optionally, the placement template can comprise a plurality of target point holders arranged spatially distributed on fixing point holder grid areas, wherein along the piercing axis. the fixing point holder grid areas are at a distance k from each other, which is also the distance of the target point grid areas from one another. By selecting one of the fixing point holders in which the fixing point is to be locked, the operator can determine in which target point grid area the applicator tip is to lie.

Optionally, the first template part can comprise a first sub-group of fixing point holders of a first fixing point holder grid area, and the second template part a second sub-group of fixing point holders of a second fixing point grid area. The two sub-groups of fixing point holders can thereby supplement each other via two fixing point grid areas of the fixing point grid structure.

Optionally, the second template part can be displaceable in the proximal direction relative to the first template part only up to the stop on the first template part, wherein when the second template part is in contact with the first template part, the first sub-group of fixing point holders defined by the first template part is located distally of the second sub-group of fixing point holders defined by the second template part.

Below, the system disclosed herein will be described in more detail by way of the accompanying figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of the operating principle of a percutaneous PDT with a light applicator without a support point element as in FIG. 23 of DE 10 2021 211 328 A1 and DE 10 2021 211 331 A1 to be published in the future;

FIG. 2 is a schematic view of the operating principle of a percutaneous PDT without a support point element as in FIG. 30 of DE 10 2021 211 328 A1 and DE 10 2021 211 331 A1 to be published in the future;

FIG. 3a and FIG. 3b are schematic views of the problem of a percutaneous PDT in the case of different positions of the organ or organ segment to be treated;

FIG. 4a and FIG. 4b are schematic views of the problem of a percutaneous PDT in the case of a relatively large volume of the organ or organ segment to be treated;

FIG. 5a, FIG. 5b, FIG. 5c, FIG. 5d, FIG. 5e, FIG. 5f, FIG. 5g, and FIG. 5h are schematic views of the problem of a percutaneous PDT in the case of piercing the skin of a patient with relatively thin light applicators;

FIG. 6 is a schematic view of an example of embodiment of a herein disclosed system for performing a transcutaneous PDT with a schematically shown support point element;

FIG. 7 is a schematic view of a further example of embodiment of a herein disclosed system for performing a transcutaneous PDT with a support point element;

FIG. 8 is a schematic view of a further example of embodiment of a herein disclosed system for performing a transcutaneous PDT with a support point element;

FIG. 9a, FIG. 9b and FIG. 9c are schematic views of a further example of embodiment of a herein disclosed system for performing a transcutaneous PDT with a support point element;

FIG. 10a and FIG. 10b are schematic views of a further example of embodiment of a herein disclosed system for performing a transcutaneous PDT with a support point element;

FIG. 11 is a schematic view of a further example of embodiment of a herein disclosed system for performing a transcutaneous PDT with a support point element; and

FIG. 12 is a schematic cross-sectional view of the system shown in FIG. 11.

### DESCRIPTION OF PREFERRED EMBODIMENTS

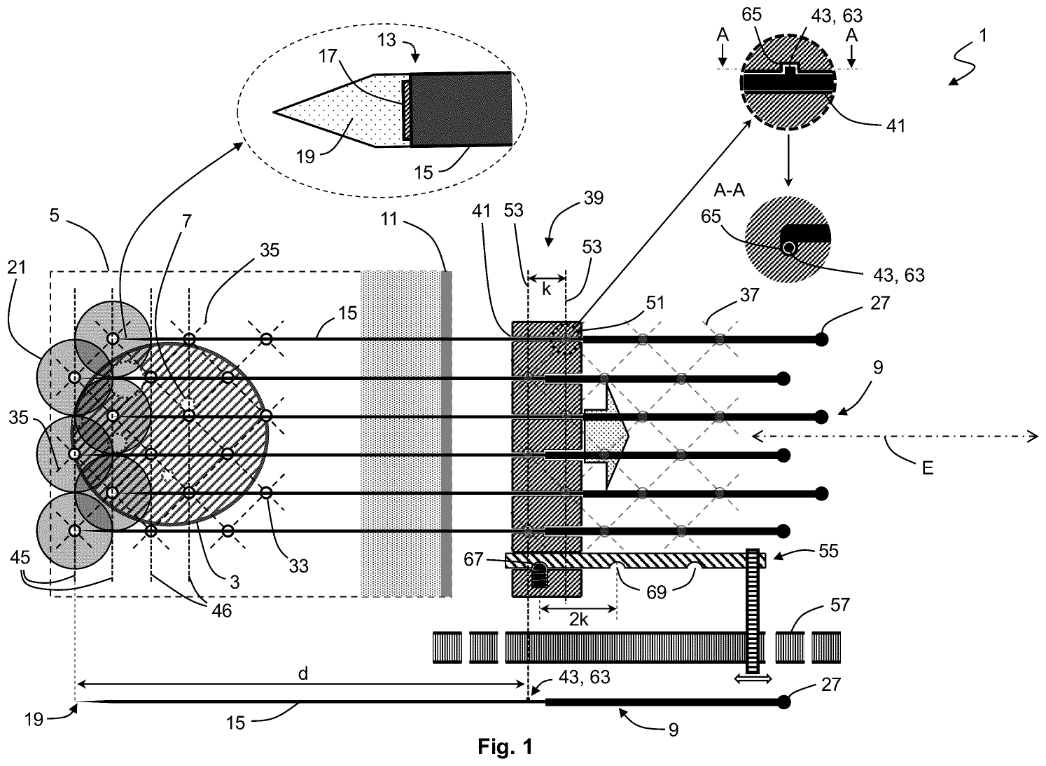

Referring to the drawings, FIG. 1 shows a system 1 for performing a transcutaneous photodynamic therapy (PDT) in an organ 3, for example the prostate, of an organic body 5. Here, the organ 3 has several pathological tissue regions 7 (shown in the figures by a dotted border) in which an administered photosensitiser or marker substance has accumulated. However, due to their small size, for example, and/or their inadequate contrast, not all pathological tissue regions 7 can be visualised and, accordingly, also due to their partial invisibility to the operator, cannot all be individually treated in a targeted manner, i.e. focally or tissue-specifically. It is known from experience though, for example, taking random biopsies from the organ 3, that such pathological tissue regions 7 are nevertheless almost always present. All the visible and invisible pathological tissue regions 7 are now to be irradiated with light in order to achieve an appropriate therapeutic effect in all pathological tissue regions 7. If certain pathological tissue regions 7, for example a tumour, are not irradiated they remain untreated and can grow or otherwise remain or become medically harmful.

In FIG. 1 it is clearly shown that the system 1 comprises a plurality of needle-like light applicators 9 which are pierced through the skin 11 of the body 5, i.e. transcutaneously, into the organ 3. The individual light applicators 9 are all identical to each other and are configured to be as thin as possible in order to do as little damage a possible to healthy tissue of the body 5 when piercing, i.e. to make a minimally invasive procedure possible. At the distal end 13 of a thin shaft-like insertion section 15 of the light applicator 9 a light-emitting element 17 in the form of an LED is arranged. Distally of the LED 17, arranged on the distal end 13 of the insertion section of the light applicator 9, is a light-transparent and light-scattering applicator tip 19. Through the applicator tip 19, the light of the LED 17 is emitted as isotropically as possible at a solid angle of over 3π. The light emission from the applicator tip 19 is thus approximately spherical, which is shown in FIG. 1 by a light sphere 21, or in the present cross-sectional drawing by a corresponding circle. The size of the light sphere 21 as an orientation or measure of in which tissue region or tissue volume a sufficiently high amount of light is still provided in view of the desired therapeutic effect, depends on the penetration depth of the light into the tissue of the organ 3 and is thus organ-specific.

The system 1 also comprises a, not shown, supply unit, with which the light applicator 9 is supplied with power, with which the LED 17 is operated. The supply unit preferably comprises a plurality of connections for the plurality of light applicators 9 which are simultaneously used in the PDT. At the proximal end of the insertion section 15, the light applicator 9 has a grip element 17 with which an operator can manually grip and position the light applicator 9. Via a cable with a plug, which fits into one of the connections of the supply unit, the light applicator 9 can be connected and supplied with power (not shown). The insertion section 15 of the light applicator 9 preferably comprises a core and a sheath electrically insulated from the core, so that the core and sheath can act as a supply and return conductor pair in order to supply the LED 17 with power. Alternatively or additionally, for this an additional conductor can be provided in the insertion section 15 of the light applicator 9.

In FIG. 1, by way of the light sphere 21 or its size, it is already clear that the penetration depth of the light is not sufficient to irradiate the entire organ 3 with one light applicator 9. However, the objective is seamless irradiation of the entire organ 3, as on the one hand in the procedure shown here, irradiation with light is not harmful for healthy tissue as due to the comparatively small amount of applied light in the PDT a destructive effect can only take place where the photosensitiser has accumulated, which because of the tumour-selectivity of the selected photosensitiser only takes place in malignant tissue, and, on the other hand, it is to be ensured that no pathological tissue region 7 and, in particular, no pathological tissue region 7 in the organ 3 that is invisible to the operator, remains untreated. After an irradiation, an operator would thus have to newly insert the light applicator 9 at other points in order to gradually, i.e. sequentially, cover the entire volume of the organ 3 with the corresponding light spheres 21.

However, such a procedure requires the operator to have very good experience and is associated with many risks and disadvantages. On the one hand it cannot be ensured that in the end the entire volume of the organ 3 has been seamlessly irradiated, as firstly the operator has many freedoms when placing the light applicators 9 in the organ 3 and because on the other hand the region that was therapeutically effectively recorded, or its boundaries, which are shown in FIG. 1 by the surface of the light sphere 21 or in the cross-sectional view by the outline of the circle, cannot be directly visualised and thereby made directly visible to the operator, so that there is no certainty about whether the light applicator 9 or its light-emitting applicator tip 19 has always been placed at the correct point. On the other hand, “poking around” can occur, where too many piercings are required which are burdensome for the patient. Finally, the PDT in this form can take a very long time.

In FIG. 1 it is shown how the PDT can be speeded up, if several individual light applicators 9 are used simultaneously. This makes the system 1 more expensive, but saves time. It makes sense to place the applicator tips 19 at all virtual target points 33 of a virtual three-dimensional target point grid structure 35. The target point grid structure 35 is matched to the shape and position of the organ 3 as well as the penetration depth of the light in the organ 3. The target point grid structure 35 is thus organ-specific. The distances between the virtual target points 33 are matched to the organ 3 in such a way that on the one hand the light spheres seamlessly overlap and the entire organ 3 is seamlessly irradiated, but on the other hand, as a patient-friendly procedure, this overlapping is only pursued as far as necessary, i.e. so that unnecessarily small distances between the light-emitting applicator tips 19 are avoided in order to thereby minimise the number of light applicators 9 or piercings by light applicators. These two opposing requirements result in a target point grid structure 35 in which the position and above all the distances of the virtual target points 33 with regard to each other are very precisely defined with comparatively narrow tolerances.

The difficulty only consists in placing the individual applicator tips 19 precisely on these virtual target points 33 as the virtual three-dimensional target point grid structure 35 is not visible to the operator and therefore the operator cannot specifically and directly approach the virtual target points 33 with the light-emitting applicator tips 19.

In a first step, the virtual and invisible target point grid structure 35 with its virtual and invisible target points 33 is represented from the inside of the body 5 on a fixing point grid structure 37 outside the body 5, which is formed by an actually existing and visible placement template 39 with actually existing and visible fixing point images 51 which the operator can specifically and directly select with the light-emitting applicator tips 19. This representation of the target point grid structure 35 on the fixing point grid structure 37 involves a parallel displacement or translation about a determined length d, which is defined by the distance from the organ 3 to the space outside the body where the placement template 39 is to be positioned, in parallel to an axis E, the direction of which is defined by the piercing axis of the applicators 9. The axis preferably, but not necessarily, runs orthogonally to the skin 11 of the patient.

Only in a later stage does the actual, therapy-preparing applicator placement then take place, in that the individual light-emitting applicator tips 19 are moved to the actually existing and visible and thus specifically and directly selectable fixing point images 51 of the placement template 39, in order to be “represented” or moved from there in a defined, clear and thus safe manner to the virtual target point 33 that are invisible to the operator. This second representation to be carried out as part of the two-stage procedure, i.e. the parallel displacement of the light-emitting applicator tips 19 from outside the body 5 into the body 5 is now, but in accordance with this procedure precisely characterised in that it is inverse to the parallel displacement of the target point grid structure 35 from inside the body 5 to outside. Specifically this means that the applicator tips 19—starting from the fixing point holders 51 and thus starting from the first representation or parallel displacement—are moved by the same defined length d and in parallel to the same axis E, but exactly counter to the first parallel displacement.

Here, the definiteness, unanimity and safety with regard to accuracy in the placement of the individual light-emitting applicator tips 19 in the target points 33 in the organ, i.e. in this case the decisive features in terms of the required seamless irradiation of the organ 3 are achieved when inserting the light-emitting applicator tips 19 from the fixing point holders 51 outside the body 5 to the virtual target points 33 inside the body 5—in spite of the target points 33 being invisible to the operator and the resulting impossibility of reaching these in a targeted manner—in that the degrees of freedom for the operator when moving the light applicators 9 or placing the light-emitting applicator tips 19 in the body are reduced to a minimum, i.e. there are no possibilities of movement other than the required parallel displacement of the light-emitting applicator tips 19 exactly inversely to the first parallel displacement of the target point grid structure 35.

Guiding the movement of the individual light-emitting applicator tips 19 into the body 5 exclusively in accordance with the inverse parallel displacement, takes place firstly in that the placement template 39 in addition to the already mentioned fixing point holders 51, is also provided with guides 41, which have a definite orientation, namely a parallel orientation to the axis E, through which only one defined direction of movement, namely the one for the desire inverse parallel displacement, of the light applicators 9 with their applicator tips 10, parallel to the axis E is permitted or is possible, and secondly in that the light applicators 9 are all provided with at least one fixing point 43, which is at a defined distance d from the light-emitting applicator tip 19 which precisely corresponds to the displacement length d of the target point grid structure 35 in the first representation or parallel displacement, and which is fixable in or on the fixing point holders 51 of the placement template 39, through which only one penetration depth, namely the one for the desired inverse parallel displacement, of the appurtenant light-emitting applicator tips 19 in the body 5 and into the organ 3 is permitted or possible.

Both together thus means that in the case of movement of the individual light applicators 9 in the distal direction to the stop in the placement template 39—starting from the visible and thus directly selectable fixing point holders 51—the light-emitting applicator tips 19 also all reach the target point 33 automatically and thereby, the desired seamless irradiation of the organ 3 is automatically guaranteed, provided that all fixing point holders 51 of the template 39 are occupied by light applicator 9 and activated.

The operator therefore only has to move the individual light applicators 9 to the fixing point images 51, then pass them through the guides 41 and pierce them transcutaneously into the organ 3 until the fixing point 43 is on the grid point of the fixing point grid structure 37, i.e. on the fixing point receiver 51. The applicator tip 19 is then also automatically placed at the corresponding target point 33. Through this, the operator has no or only a few degrees of freedom and does not require great experience. Furthermore, placement can be carried out quickly with just as many punctures as necessary but as few as possible. At the same time, the PDT can be performed with all placed light applicators 9, which makes a short treatment duration possible.

This procedure is therefore based on implementing two representations, namely on the on the other hand the representation of the target points 22 on the fixing point holders 51 of the placement template 39, and, on the other hand, the representation of the light-emitting applicator tips 19 starting from the fixing point holders 51 back to the target points 33, wherein the second representation is characterised in that it is inverse to the first representation, and secondly in that it is mechanically ensured that the second representation, which corresponds to the actual placement of the applicators by the operator, has no alternative, i.e. a movement of the applicators 9 or the applicator tips 19 deviating from the second representation—starting from the fixing points or fixing point holders 51 in the body 5 and in to the organ 3—is not possible at all.

With increasing size of the organ 3, the number of required light applicators 9 grows very greatly. For example, double the diameter of a spherical organ 3, means that eight times as many light applicators 9 are needed. In addition, if on the other hand, the penetration depth of the light is comparatively low and, on the other hand, the extent of the organ 3 along the piercing axis E is relatively large, the density of the light applicators 9 in the planes perpendicular to the piercing axis E is correspondingly high and it can happen that the light applicators 9 no longer have sufficient space next to each other.

This problem can be countered by performing a PDT in stages. In FIG. 1 it is shown how the PDT can be performed in stages or successively in the target point grid areas 45, then 46 etc. of the target point grid structure 35. For this, the fixing point 43 of the light applicators 9 are placed or fixed at the grid points or the fixing point holders 51 of an appurtenant fixing point grid area 53 so that the individual applicator tips 19 are placed at the corresponding target points 33 of the target point grid area 45. The PDT (as an example in FIG. 1) is performed for the first two target point grid areas 45, which are preferably the target point grid areas 45 lying deepest under the skin, i.e. furthest distally. The light applicators 9 can then be retracted proximally by a distance 2k in order to perform the PDT for the next two target point areas 46. This is carried out in stages for all target point area pairs 45, 45 etc. until the entire volume of the organ 3 has been irradiated with light. In FIG. 1, the placement template 39 therefore forms, for example, two fixing point grid areas 53 offset in parallel to each other at a distance k, so that two adjacent target point grid areas 45 can be irradiated at the same time. The light applicators 9 can then be retracted proximally by a distance 2k of the target point grid areas 45 in order to perform the PDT for the next two target point grid areas 45. Advantageous in this stage-by-stage procedure, is the fact that fewer light applicator 9 and fewer piercings are required as well as the treatment time being reduced.

In FIG. 1, the placement template 39 can be displaced as a whole by the distance 2k in relation to a fixed reference surface 57. By way of the fastening device 55, the placement template 39 defines several fixing points arranged at a distance 2k, which are configured as engagement receptacles 69 in the fastening device 55 in order to be able to displace the fixing point holder areas 53 as a whole by the distances 2k. This has the advantage that when changing to the next target point grid area 46, the light applicators 9 do not have to be moved individually, but can be moved concerted together. This speeds up and simplifies the treatment process. After the PDT with the applicator tips 19 in the first and second target point grid area 45, the organ 3 is irradiated with the applicator tips 19 in the third and fourth target point grid area 46.

In FIG. 1, the placement template 39 is a block or otherwise shaped body with a certain thickness along the piercing axis E, through which a plurality of guides 41 arranged in parallel runs along the piercing axis E. Each guide 41 has a fixing point holder 51 which forms a grid point of the fixing point grid structure 37. The thus formed three-dimensional fixing point grid structure 37 is identical to the organ-specific virtual fixing point grid structure 35 for the applicator tips 19 and comes about from this through parallel displacement or translation by length d. Each light applicator 9 has a thin insertion section 15 and a thicker proximal section that serves as a grip element 27 as well as for guiding in the guides 41. The guides 41 are dimensioned in accordance with the diameter of the insertion section 15 and the thicker proximal section of the light applicators 9. The light applicators 9 are all identical in order to prevent the risk of incorrect selection of a “wrong” light applicator 9 and the thereby resulting incorrect positioning of the light-emitting applicator tip 19 in the organ 3. The operator has no degrees of freedom here, but can only insert the light applicators 9 in one way until the fixing points 53 engage in the fixing point holders 43. For this, the fixing point holders 51 can have jaw clips or, as shown in FIG. 1, detent lugs or other clamping means. In FIG. 1, the fixing points 43 and the fixing point holders 51 form, for example, a type of bayonet closure, in which a lateral projection 63 on the light applicator 9 through turning the light applicator 9 about the piercing axis E can be turned into a corresponding lateral securing device 65 in the guide 41. This give the operator haptic feedback on the corrected placement and prevents unintentional proximal slipping of the light applicators 9. The placement template 39 is moveable in a guided manner along the piercing axis E by way of its fastening device 55 and is lockable relative to the reference surface 57. The position of the placement template 39 in relation thereto can preferably be determined as follows. The placement template 39, with the fastening device 55 is initially placed and fixed on the reference surface 57 very close to the body 5 or on the skin 11. Thereafter, a first light applicator 10, preferably to be positioned centrally in relation to a plane perpendicular to the piercing axis E and distally in relation to the piercing axis E itself is pierced in and—monitored by means of an imaging method (for example ultrasound sonography)—advanced so far until its applicator tip 19 had reached the distal area of the organ 3. Then—while maintaining this achieved position of the first light applicator 10 or its applicator tip 19—the placement template 39 is pulled back by means of the fastening device 55 on the reference surface 57 axially, i.e. along the piercing axis E, so far in the proximal direction away from the skin 11, until the fixing point holder 43 can be fixed in the fixing point holder 51. In this position the placement template 39 is then fixed on the reference surface 57 by means of the fastening device 55.

With this action, the defined distance d between the virtual target point grid structure 35 and the fixing point grid structure 37 is brought about. This action thus completes the first step of the procedure used here, namely the first representation, i.e. the parallel displacement or translation of the virtual target point grid structure 35 in the organ 3 inside the body into an area outside the body 5 and thus creates the precondition for the actual preparation of the therapy, namely the correct placement of the remaining light applicators 9 or their light-emitting applicator tips 19 in the organ 3.

The remaining light applicators 9 then only have to be passed through the remaining guides 41 of the placement template 39 and fixed with their fixing points 43 to the respective fixing point holder 51. The degrees of freedom of the operator are thereby minimised, which in turn means that the positions of the remaining light applicators 9 and consequently the positions of their light-emitting applicator tips 19 after fixing of the placement template 39 are clearly determined and thereby automatically correspond with the target point 33.

The principle, shown in FIG. 1, of treating several target point grid areas 45 simultaneously, mainly becomes more important if the extent of the organ 3—above all also along the piercing axis E—is comparatively large, as through this, at the same time or with the same number of sequential irradiation units (in this case three irradiation units), a considerably greater volume can be treated. For this, the placement template 39 has fixing point holders 51 of two adjacent fixing point holder grid area 53. The placement template 39 can then be displaced by double the length, i.e. by 2 k in order to perform the PDT for the following two target point grid areas 45. With this procedure, i.e. that with one irradiation unit all target points from several target point grid areas 45 are covered simultaneously, in the same time, i.e. with the same number of irradiation units, larger, i.e. more greatly extended organs 3 along the piercing axis E, can be treated. The fastening device 55 of the placement template 39 comprises an engagement receptacle element 67 which engages in engagement receptacles 69, which in relation to the reference surface 57 are firmly arranged along the piercing axis E each at a distance of 2 k from each other.

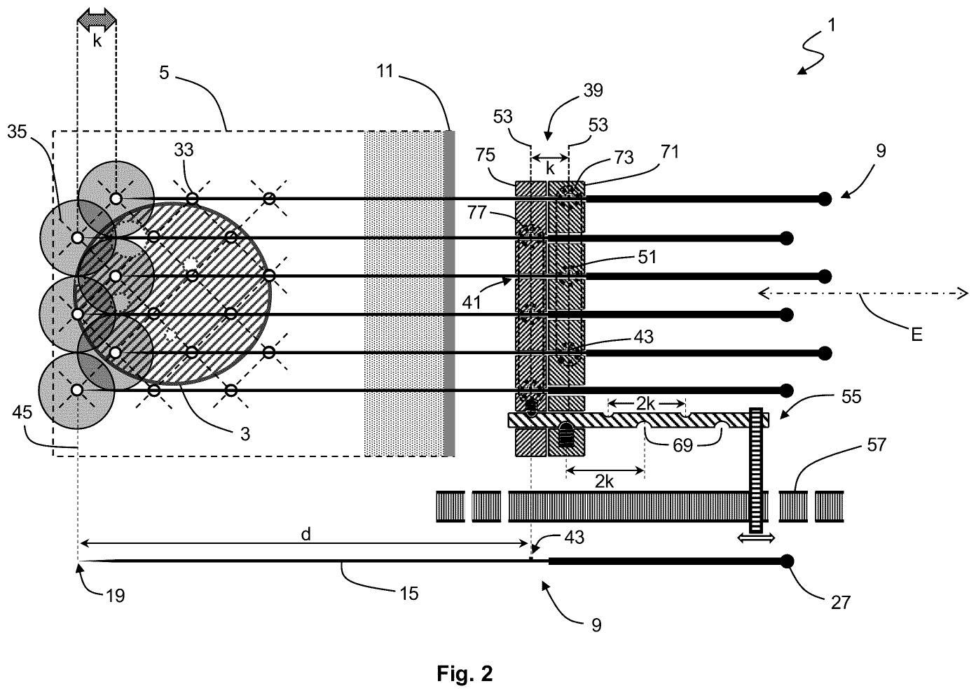

FIG. 2 shows a solution principle for the “stacking problem” in which the individual light applicators 9 pierced into the organ 3 on proximal pulling back pull back the organ and/or deform or extend/stretch the organ 3. The placement template 39 here comprises at least two parts, namely a first template part 71, which defines a first sub-group 73 of the guides 41 with fixing point holders 51, and a second template part 75 which defines a second sub-group 77 of guides with fixing point holder 51. The first template part 71 is here displaceable relative to these second template part 75 in a guided manner along the piercing axis E. By way of the template parts 71, 75, various sub-groups of light applicators 9 can, through the corresponding sub-groups 73, 77 of guides with fixing point holder 51 can be optionally concertedly jointly moved or held in position. This has the advantage that within one group of light applicators 9 the organ 3 can be held fast while the other sub-group is moved. The “stacking effect” can be also be avoided or at least reduced by way of one sub-group of light applicators being held 9 fast while the other is being pulled out. The respective sub-groups can be alternately moved, while the other is held fast. The joint movement and joint holding of the light applicators 9 can be brought about by corresponding movement or holding of the template parts 71, 75. The first template part 71 with the first sub-group of light applicators 9 can be moved proximally while the second template part is held in place. The sum of the static frictional forces, which are exerted by both the moved light applicators 9 of the first sub-group 73 as well the held light applicators 9 of the second sub-group 77 on the organ, in a first approximation is zero. As a result, with this procedure it is possible to move a large number of light applicators 9 jointly and/or simultaneously in a joint direction, here in a proximal direction, without entraining the organ 3 and/or deforming the organ 3.

The second template part 75 with the second sub-group 77 of light applicators 9 can be pulled proximally while the first template part 71 with the first sub-group 73 of light applicators 9 is held fast. The sum of all static frictional forces jointly exerted by all light applicators 9 on the organ 3 is here also zero in a first approximation, so that in spite of the simultaneous movement of a large number of light applicator 9, namely those of the second sub-group 77, in a joint direction, the organ 3 is neither entrained or deformed.

The comparatively high density of guides 41 and fixing point holders 51 and the resulting high potential density of applicators 9 in the organ 3, as well as the arrangement of the guides 41 and fixing point holders 51 of different sub-groups 73 and 77 and accordingly the applicators 9 of different sub-groups 73 and 77 in relation to each other, i.e. the regular change of belonging of guides 41 and fixing point holders 51 to the different sub-groups 73 and 77, means that local displacements and deformations of the organ 3 can also largely be avoided.

In FIG. 2, the placement template has two parallel plates 71, 75 which form the template parts 71, 75 and are moveable relative to each other guided along the piercing axis E. The first template part 71 has the fixing point holders 51 of a first fixing point holder grid area 53 and the second template part 75 has the fixing point holders 51 of a second fixing point holder grid area 53.

If a “stacking effect” is feared, the first template part 71 can initially be pulled back by 2 k while the second template part 75 is held in place. As soon as the first template part 71 is positioned, the second template part 75 can be pulled behind proximally by 2 k, while the first template part 71 is held fast.

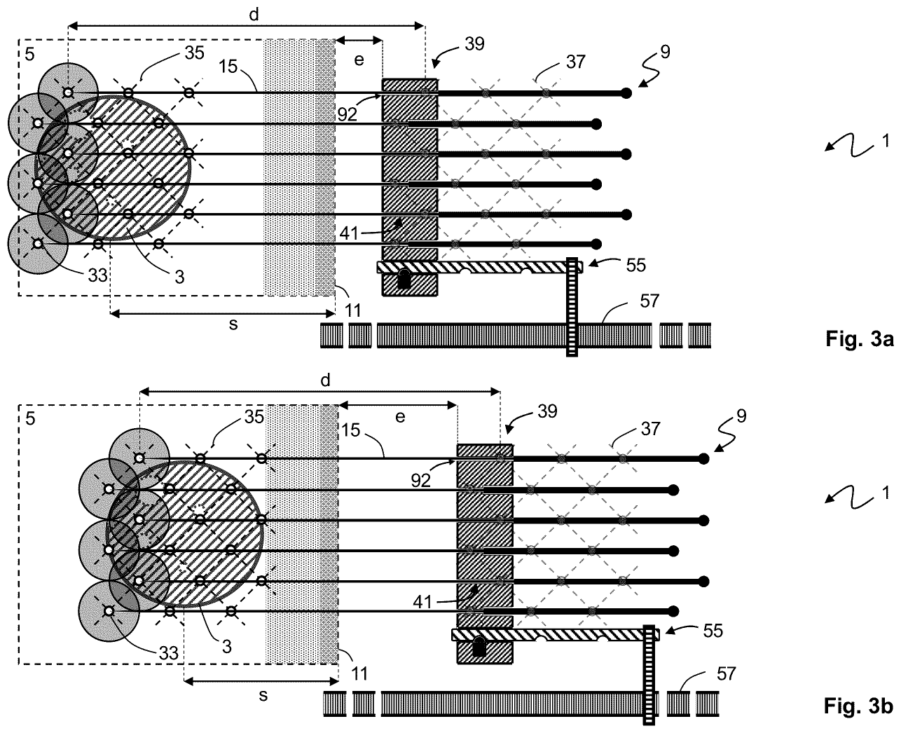

FIGS. 3a, b shows the system shown in FIG. 1 in various cases of use. In FIG. 3a, the organ is relatively far away from the skin 11 of the body 5 of the patient. In FIG. 3b, the organ is closer to the skin 11 of the body 5 of the patient. As for the many different cases of use, versions of light applicators 9 of different lengths do not want to be stocked and accordingly correctly selected, a distance e between the skin 11 and placement template 39 must be adjusted in such a way that the fixing point grid structure 37 is correctly placed, so that target point grid structure 36 parallel displaced by distance d with regard thereto is correctly positioned in the organ 3. As has already been described above, this is achieved by way of corresponding positioning of the fastening device 55 in relation to the reference surface 57 connected to the body 11. The body 11 can, for example, be fixed on a treatment table, which defines the reference surface 57.

As shown in FIG. 3b, the distance e can be relatively large if the organ 3 is relatively close to the skin 11. The distance d determined by the light applicators 9 is conceived for organs 3 lying at a maximal depth under the skin 11, see FIG. 3a, in order to still be able to reach and treat them with the same light applicators 9. Consequently, there is a correspondingly greater distance e in the case of organs located closer to the skin 3 as shown in FIG. 3b.

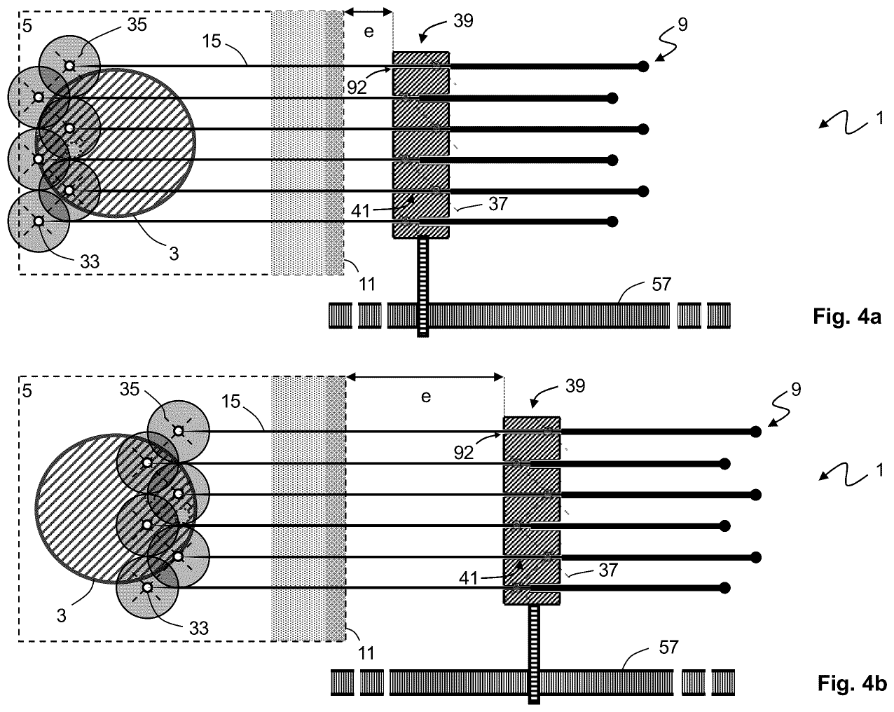

In FIGS. 4a,b it is clear that a greater distance e can result not only in a different case of use, but also during a case of use. This is the case if the organ 3 is of a certain length along axis E. After each irradiation step in one or more target point grid areas 45, the placement template is after all moved proximally so that the distance e gradually increases. Thus, if the organ 3 extends to relatively close to the skin 11, the distance e for the treatment of the proximal end region of the organ 3 becomes relatively large. If, for example, in the proximal organ area a higher applicator density is needed as, for example, there is or is assumed to be more pathological tissue there, with a relatively large distance e, further light applicators have to be pierced.

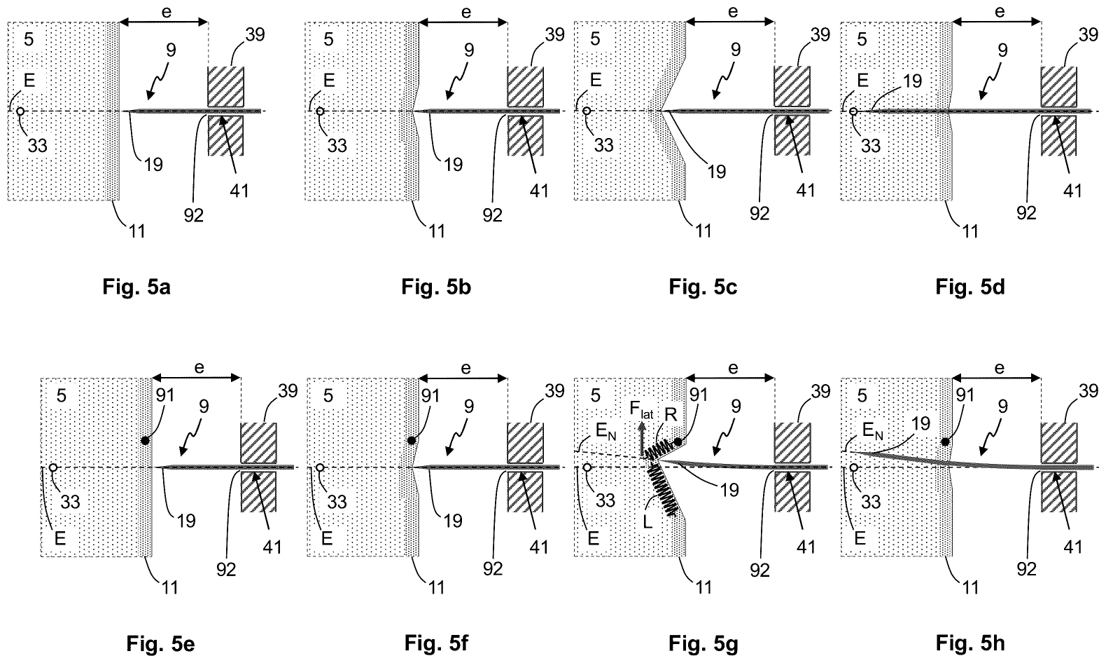

FIGS. 5a-h show a problem when piercing the skin in the case that the distance e between the skin 11 and the distal end 92 of the guide of the placement template 39 is relatively large, as in FIGS. 3b and 4b for example. Shown in FIGS. 5a-d is a problem-free piercing of the skin in which the applicator tip 19 moves precisely along the piercing axis E and accurately reaches the target point 33 in the organ 3. Among other things, this is due the fact that both the skin 11 as well as the subsequently pierced tissue in the body 5 is homogenously structured. Through this, on introducing the light applicator 9 through the skin 11 into the body 5 in the axial direction only such a resulting force can act back on the light applicator 9 that exclusively has an axial component, but not a side or lateral component Fₗₐₜ.

However, in FIGS. 5e-h, the skin exhibits inhomogeneity 91 in the structure of the tissue. This could be caused by a scar, proximity to bone or cartilage, a growth, generally modified fibre density or other disturbance with regard to homogeneity. As in FIGS. 5a-d, here too a force F reacts to the light applicator 9 that pierces the skin 11 and penetrates into the body, thereby exerting a force on the tissue. As clarified in a spring model in FIG. 5g, in the case, due to the inhomogeneity 91, this involves a resulting force which also has a side or lateral component Fₗₐₜ. This side or lateral force Fₗₐₜ acts as a bending force on the light applicator 9 and can thereby cause buckling thereof. Decisive for the dimension or strength of the resulting light applicator bending is the bending moment M, which is formed by the product of bending force, i.e. the lateral force component Fₗₐₜ of the resulting force acting back on the light applicator 9, and remaining light applicator length, i.e. the distance e between skin 11 and placement template 39. However, in turn this means that with increasing distance e, the dimension or strength of the resulting light applicator buckling continues to increase further. Secondly, this means that even a small lateral force Fₗₐₜ can bring about comparatively great buckling of the light applicator 9 or its applicator tip 19 if the distance e is sufficiently great, as shown in FIG. 5g, h. Very specifically, however, this means that even slight and thus apparently insignificant inhomogeneities 91 in the tissue structure in the area of the skin 11 can very substantially contribute to buckling of the light applicator 9, especially when the placement template 39 is not in the immediate vicinity of the body 5 or the skin 11. Furthermore, if, in order to protect the patient, the light applicator 9 is made very thin, then the buckling is correspondingly greater or even comes about at correspondingly shorter distances e. However, such buckling results in the applicator tip 19 no longer being moved along the desired piercing axis E, but along another axis E_{N} which no longer leads to the target point 33. The light-emitting applicator tip 19 would then, after completed placing of the applicator 9, no longer be on the target point 33 as planned and during irradiation organ regions could remain unirradiated or at least insufficiently irradiated.

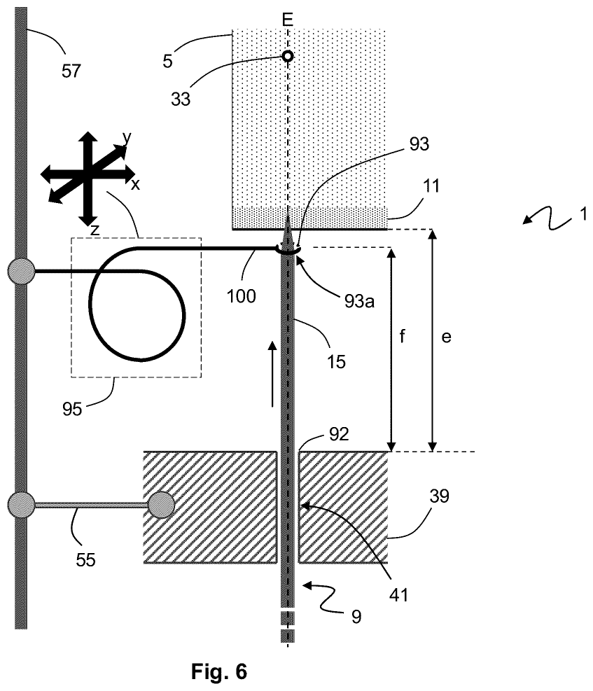

FIGS. 6 to 12 show both schematic views of the principle of the procedure as well as specific forms of embodiment of the invented solution to this problem. A support point 93 for the light applicator 9 that is adjustable between the skin 11 and the placement template 39 and thus positionable as desired, shortens the distance e, effective for bending, by a distance f of the support point 93 from the placement template 39. Through this is becomes possible to decisively reduce or minimise a bending moment potentially acting on the applicator tip 19 (caused by at least one inhomogeneity 91 in the region of the skin), which is the decisive parameter for the dimension or severity of the buckling of the light applicator 9 during its insertion and which thereby essentially determines the precision of the applicator tip 19 in relation to the reached target point 33 during placement of the applicator The support point 93 is here formed by an opening 93 in a support point element 100, through which the respective light applicator 9 can be passed in a precisely fitting manner, The support point element 100 thus defines for each of the light applicators 9 a support point 93 positioned or positionable coaxially to the piercing axis E for the lateral supporting of the respective light applicator 9, wherein the distal distance f of the support point 93 from the placement template 39 is adjustable, and can be maximised for the purpose of minimising a potential bending moment on the light applicator tip 19 when introducing the light applicator 9 into the body 5.

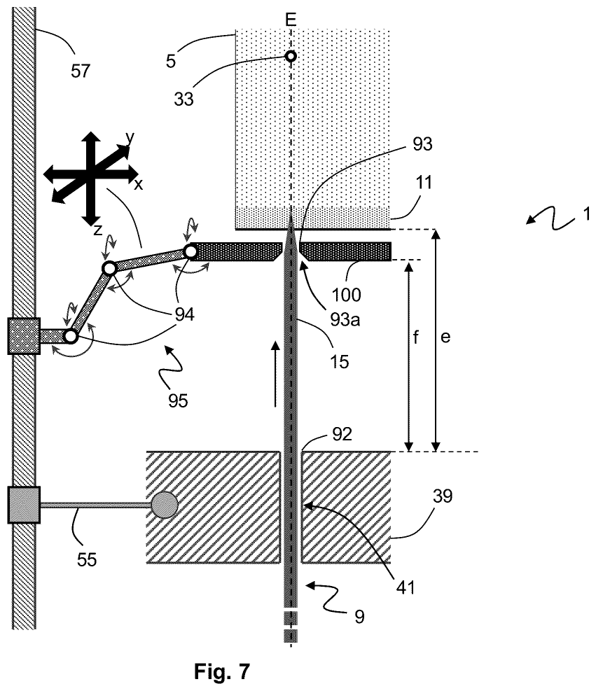

Characteristic of the procedure shown in principle in FIG. 6 and its concrete example of embodiment shown in FIG. 7, is that the support point element 100 is not only moveably supported and lockable in position relative to the placement template 39 along the piercing axis E, but also in an xy plane extending perpendicularly thereto. For this, the support point element 100 is connected to the reference surface 57 by means of an adjusting component 95, adjustable via joints 94 in three spatial directions x,y,z, which in the principle drawing of FIG. 6 is shown schematically and symbolically as a flexarm or swan neck. The support point 93 of the support point element 100 can then be positioned in the xy plane flush with the guide 41 of the placement template 39 and locked. In the z direction, i.e. along the piercing axis E, as large a distance f as possible can be set, so that the light applicator 9, through minimising a potentially active bending moment on the applicator tip 19—caused by potential inhomogeneities 91 in the region of the skin 11—is protected as well as possible against bending during piercing into the skin 11.

FIG. 7 shows a specific example of embodiment of the principle procedure shown in FIG. 6. Here, the adjusting component 95 is configured as an articulated arm with joints 94, which for maximum freedom of movement can, at least in parts or all be in the form of ball joints. The support point element 110 is configured as a perforated plate which is directly connected to the articulated arm 95. The opening 93a is a hole in the perforated plate, which is additionally provided with an insertion slope so that the light applicator 9 can be passed through better.

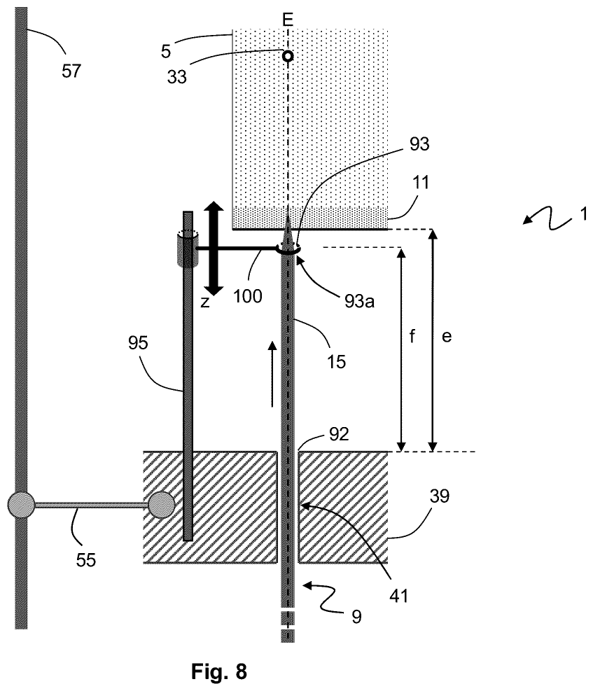

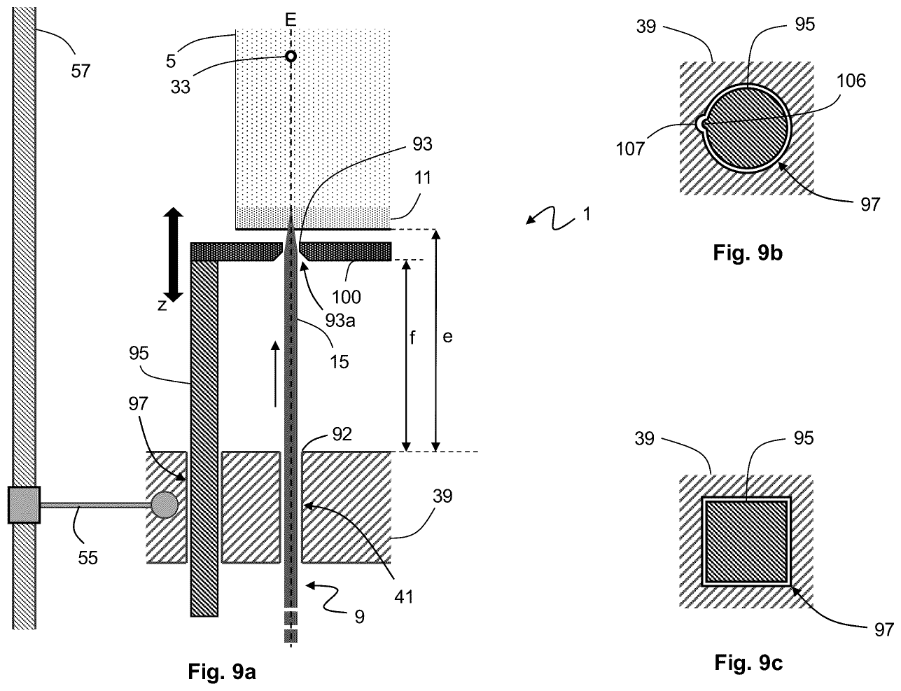

Characteristic of the procedure shown in principle in FIG. 8 and its concrete example of embodiment shown in FIGS. 9a-b, is that the freedom of movement of the support point element 100 relative to the placement template 39 is restricted to the z axis, i.e. the piercing axis E. Here, the adjusting component 95 is not directly connected to the reference surface 57, but only indirectly via the placement template 39, which is connected to the reference surface 57. In the procedures shown in FIGS. 8 and 9a-c, only the distance f for the support point element 100 vis-a-vis the placement template 39 can therefore be adjusted, preferably to a maximum. The coaxial orientation of the support point 93 in relation to the guide 41 is determined structurally or by configuration and does not have to adjusted by an operator. On the one hand this reduces the work for the operator, and on the other hand eliminates sources of error when positioning the support point element 100 or the support point 93. In the example of embodiment in FIGS. 9a-c, the adjusting component 95 is configured as an elongated rod element, which is guided in recess 97 of the placement template 39. In accordance with FIG. 9b, the adjusting component 95 is secured against turning about the z axis by a male projection 106 which engages in a corresponding female recess 107 in the holder 97 of the placement template 39. It goes without saying that the male projection 106 can alternatively be arranged on the holder 97 of the placement template 39 and the female recess 107 on the adjusting component 95 accordingly. In FIG. 9c, securing against turning is achieved by a non-circular, here rectangular or quadratic cross-section of the adjusting component 95.

In the case of the procedure shown in principle in FIG. 8 and the concrete form of embodiment in FIGS. 9a-c, it is the placement template 39 which is connected via the fastening device 55 to the reference surface 57, while the adjusting component 95—in contrast to the above-described procedure—is no longer directly connected to the reference surface 57, but instead has a direct and moveable connection with the placement template 39. This means that the adjusting component 95 can be moved alone, namely along one axis, but that when the placement template 39 is moved the adjusting component 95 always moves with it. This connection principle and the resulting dependency of the various components on and with regard to each other is rational if initially the placement template 39 is to be brought into the correct and final distance from the body 5 (bringing about of the distance d between the placement template and virtual target point grid structure within the body 5) and only thereafter is the support point element 100 or its support point(s) 93 to be brought into the final position, i.e. close to the skin 11. In the event that initially the support point element 100 with its support point(s) 93 is to be brought into the final position, i.e. close to the skin 11, and thereafter final placement of the placement template 39 is to take place, a different connection and resulting dependency relationship of the various components on and with regard to each other is rational (no illustration). The adjusting component 95 is connected via a fastening device 55 directly to the reference surface 57, while the placement template 39 now no longer has a direct connection to the reference surface 57, but, instead, via an adjusting component 95 has a direct moveable connection. This means that the placement template 9 can be moved alone, namely along one axis, but that when the adjusting component 95 is moved the placement template 39 always moves with it.

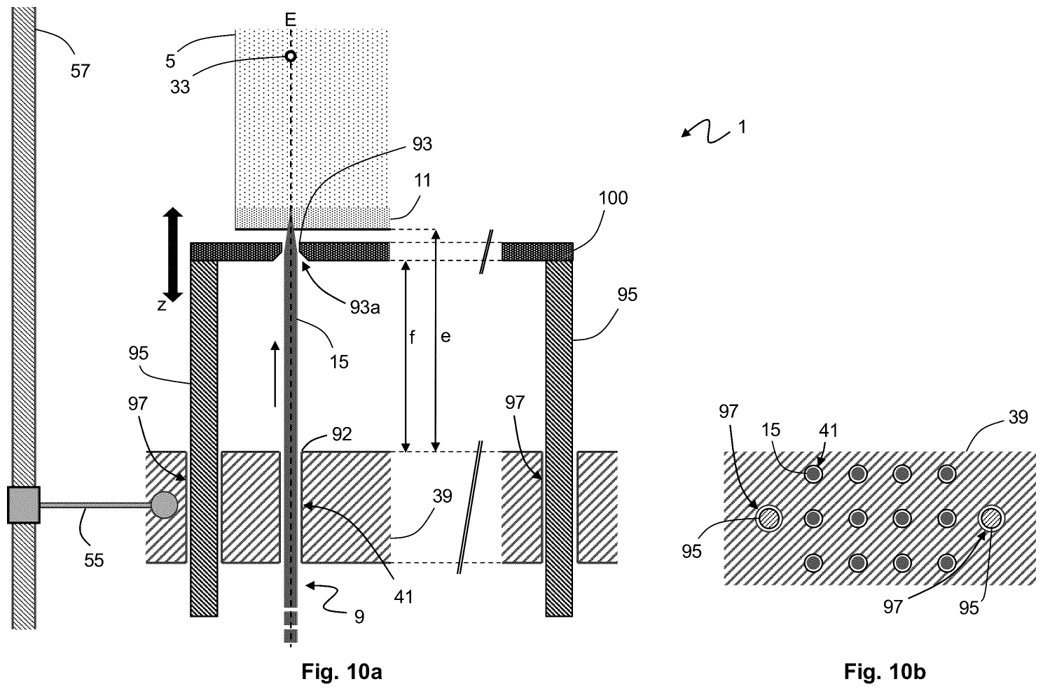

In FIGS. 10a,b the support point element 100 is in the form of a perforated plate with holes 93a, wherein each hole 93a defines a support 93, wherein the holes 93 are arranged or arrangeable coaxially to the guides 41 of the placement template 39. Here, the adjusting component 95 is configured in the form of elongated rod elements guided in holders 97 of the placement template 39 which run in parallel to each other in the z direction, i.e. the piercing axis E. As in FIGS. 8 and 9, in this way the support point element 100 can only be positioned in the z direction relative to the placement template 39 in order to set a desired maximum distance f between the support point element 100 and the distal end 92 of the guides 41 of the placement template 39, or a minimum distance between the support point element 100 and the skin 11. As shown in FIG. 10b, arranged in the placement template 39 in an xy plane perpendicular to the piercing axis E is a grid of guides 41 between the holders 97 through which the light applicators 9 can be passed with their insertion sections 15.

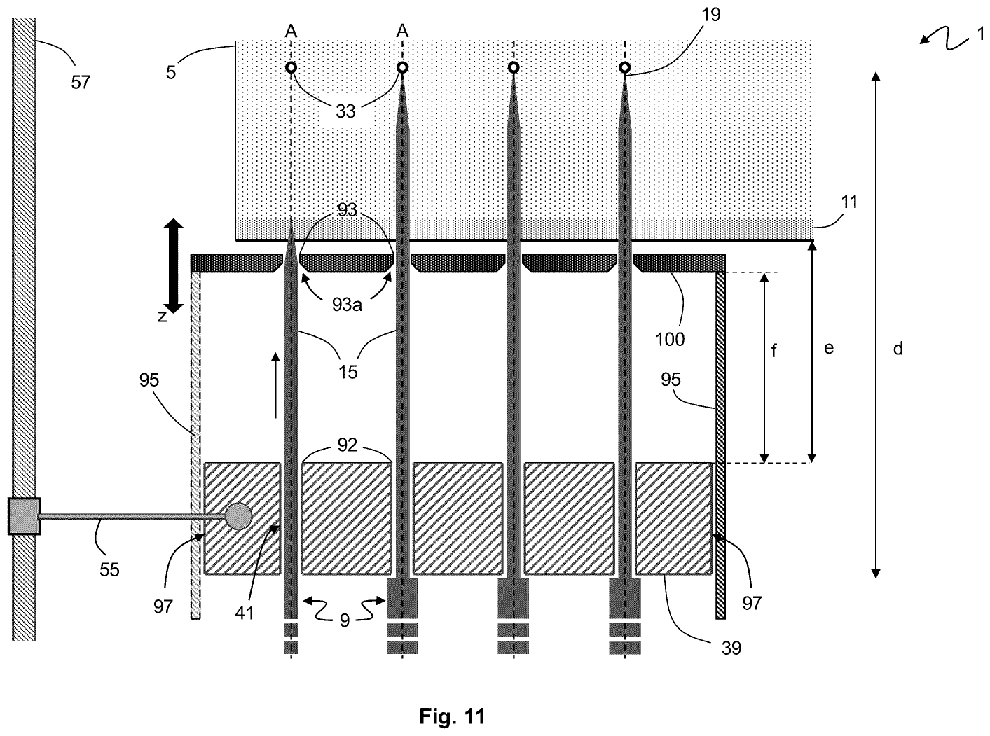

FIG. 11 shows an embodiment of the system 1 according to the invention during the performance of a transcutaneous photodynamic therapy (PDT) in an organic body 5, after the distance f of the support point element 100 from the distal end 92 of the guides 41 of the placement template 39 has been set and the first three light applicators 9 have already been finally placed with their applicator tips 19 on the target points 33 in the body 5. The fourth light applicator 9 is just being pierced into the skin 11. Through the distance f and thus the support point element 100 being positioned close to the skin 11, the risk of bending of the light applicators 9 when piercing the skin is considerably reduced.

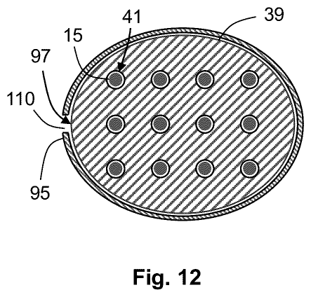

In contrast to the examples of embodiments according to FIGS. 10a-c, the adjusting component 95 is here not in the form of elongated rod elements guided in holders 97 of the placement template 30, but in the form of a tubular element that circumferentially surrounds the placement template 39. A lateral outer wall of the placement template 39 then forms the holder 97. FIG. 12 shows that securing against rotation can be ensured by, for example, an elliptical or other non-circular cross-section. A lateral slit 110 in the tubular adjusting component 95 is provided so that the fastening device 55 can project through laterally in order to be able fix the placement template 39 independently of the position of the support point element 100 in relation to the reference surface 57.

The embodiment shown in FIGS. 11 and 12 is configured so that initially the placement template 39 is placed and fixed, namely in such a way that the fixing point grid structure 37 is outside the body 5 at distance d to the virtual target point grid structure 35 inside the body, and only then does final placement template of the adjusting component 95 take place, preferably close to the skin 11. In this form of embodiment too, the inverse procedure is conceivable, i.e. initially the adjusting component 95 is finally placed (close to the skin 11) and only then is the placement template 39 finally placed. In this modified procedure, the fastening component 55 from FIG. 11 then preferably connects the reference surface 57 with the support point element 110, e.g. with the tubular adjusting component 95 and no longer with the placement template 39. The lateral slit 110 in the tubular adjustment component 95 can then be dispensed with. The structure for this modified procedure is not shown.

While specific embodiments of the invention have been shown and described in detail to illustrate the application of the principles of the invention, it will be understood that the invention may be embodied otherwise without departing from such principles.

### LIST OF REFERENCE NUMBERS

1 System
3 Organ
4 Surrounding tissue, directly connected to the organ 3
5 Body
7 Pathological tissue area
9 Light applicator
10 First light applicator
11 Skin
13 Distal end of the insertion section
15 Insertion section of the light applicator
17 Light-emitting element/LED
19 Applicator tip
21 Light sphere
27 Grip element
33 Target point
35 Target point grid structure
37 Fixing point grid structure
39 Placement template
41 Guide
43 Fixing point
45 Target point grid area
46 Target point grid area
47 Fixing point grid area
51 Fixing point holder
53 Fixing point holder area
55 Fastening device
57 Reference surface
59 Engagement element
61 Engagement receptacle
63 Projection
65 Securing device
67 Engagement element
69 Engagement receptacle
71 First template part
73 Sub-group of guides or light applicators
75 Second template part
77 Sub-group of guides or light applicators
91 Inhomogeneity
92 Distal end of the guide of the placement template
93 Support point
93a Opening in the support point element
94 Joints
95 Adjusting component
97 Holder
100 Support point element
106 Male projection
107 Female recess
110 Slit

What is claimed is:

1. A system for performing a transcutaneous photodynamic therapy (PDT) in an organ or an organ segment of an organic body, the system comprising:
a plurality of light applicators;
a supply unit for supplying the light applicators with light and/or power;
a placement template, placeable in a defined position relative to the organic body for a defined alignment, positioning and fixing of the light applicators; and
a support point element,
wherein the individually placeable light applicators each comprise a needle shaped insertion section for transcutaneous piercing along a piercing axis into the organ or organ segment, a light-emitting applicator tip at the distal end of the insertion section and at least one defined fixing point at a proximal distance d from the light-emitting applicator tip,
wherein the distance d is identical for each of the individual light applicators,
wherein the support point element for each of the individual light applicators defines, with the piercing axis, a coaxially positioned or positionable support point for a lateral supporting of the respective light applicator, and
wherein a distal distance f of the support point from the placement template is adjustable.

2. The system according to claim 1, wherein the support point is formed by an opening in the support point element, though which the respective light applicator can be passed precisely fittingly.

3. The system according to claim 1, wherein at least along the piercing axis:
the support point element is moveably borne relative to the placement template and is lockable upon being positioned; and/or
the piercing axis placement template is moveably borne relative to the support point element and is lockable upon being positioned.

4. The system according to claim 1, wherein:
the support point element is connected to the placement template moveably borne such that the support point element is only moveable linearly along the piercing axis relative to the placement template; and/or
the placement template is connected to the support point element moveably borne such that the placement template is only be moveable linearly along the piercing axis relative to the support point element.

5. The system according to claim 1, wherein the support point element has a perforated plate configuration with holes, wherein each hole defines a support point, wherein the holes are arranged or are arrangeable coaxially to light applicator holders of the placement template.

6. The system according to claim 1, wherein the support point element has a skin application surface facing away from the placement template, wherein the skin application surface can be placed on the skin of an organic body and positionably fixed there.

7. The system according to claim 1, wherein:
the support point element is also positionally movable relative to the placement template in a plane perpendicular to the piercing axis; and/or
the positioning template is positionally movable relative to the support point element in a plane perpendicular to the piercing axis.

8. The system according to claim 1, wherein the light applicators are individually insertable into the placement template and the bearing point element independently of each other.

9. The system according to claim 1, wherein the placement template comprises at least one first template part, which defines a first sub-group of the fixing point holders, and comprises at least one second template part, which defines a second sub-group of the fixing point holders, wherein the first template part is displaceable relative to the second template part guided along the piercing axis.

10. The system according to claim 9, wherein the first sub-group of fixing point holders defines a first fixing point holder partial grid and the second sub-group of fixing point holders defines a second fixing point holder partial grid wherein perpendicularly to the piercing axis the second fixing point holder partial gird is offset with regard to the first fixing point holder partial grid by a lateral grid offset.

11. The system according to claim 9, wherein a first sub-group of the fixing point holders, the second sub-group of fixing point holders as well as the light applicators together define a three-dimensional fixing point grid structure, wherein the fixing point grid structure corresponds to a virtual organ-specific target point grid structure for the light-emitting applicator tips in the organ or organ segment and is parallel displaced to the target point grid structure by a distance d along the piercing axis, wherein each light applicator has at least one defined fixing point at distance d from the light-emitting applicator tip.

12. The system according to claim 9, wherein the second template part is displaceable in the proximal direction relative to the first template part only up to the stop on the first template part, wherein on contacting of the second template part on the first template part the first sub-group of fixing point holders defined by the first template part lies distally of the second sub-group of fixing point holders defined by the second template part.

13. The system according to claim 9, wherein the first template part comprises a first sub-group of the fixing point holders of a first fixing point holder grid area and the second template part comprises a second sub-group of the fixing point holders of a second fixing point holder grid area.

14. The system according to claim 1, wherein the supply unit is configured to supply the light applicators with power and at the distal end of the insertion section and each light applicator has a power-operated LED.

15. The system according to claim 1, wherein the light applicators each have at least one fixing point which is formed by a stop and is lockable.
